(19) United States
(12) Patent Application Publication
(10) Pub. No.: US 2021/0223270 A1
(43) Pub. Date: Jul. 22, 2021

(54) METHOD FOR DIAGNOSING INTERSTITIAL CYSTITIS

(71) Applicants: Public University Corporation Nara Medical University, Kashihara-shi, Nara (JP); For You Medical Corporation, Nakagyo-ku, Kyoto-shi, Kyoto (JP); TOMO CO., LTD., Nakagyo-ku, Kyoto-shi, Kyoto (JP)

(72) Inventors: Kazumasa TORIMOTO, Kashihara-shi, Nara (JP); Kiyohide FUJIMOTO, Kashihara-shi, Nara (JP); Tomohiro UEDA, Nakagyo-ku, Kyoto-shi, Kyoto (JP)

(73) Assignees: Public University Corporation Nara Medical University, Kashihara-shi, Nara (JP); For You Medical Corporation, Nakagyo-ku, Kyoto-shi, Kyoto (JP); TOMO CO., LTD., Nakagyo-ku, Kyoto-shi, Kyoto (JP)

(21) Appl. No.: 17/263,697

(22) PCT Filed: Nov. 5, 2019
(86) PCT No.: PCT/JP2019/043302

§ 371 Date: Jan. 27, 2021

(30) Foreign Application Priority Data
Dec. 27, 2018 (JP) 2018-243996

(51) Int. Cl. G01N 33/92; G01N 30/72

(52) U.S. Cl. CPC G01N 33/92; G01N 2030/8818; G01N 30/7233

(57) ABSTRACT

A method for diagnosing interstitial cystitis includes measuring at least any one kind of lysophosphatidylcholine. A system and program for diagnosing a possibility of interstitial cystitis or bladder pain syndrome is also provided.

### TECHNICAL FIELD

The present invention relates to a method for diagnosing interstitial cystitis.

### BACKGROUND

Interstitial cystitis is a “disease that is associated with non-specific chronic inflammation of the bladder and presents symptoms such as frequent urination, increased desire to urinate, urinary urgency, and bladder pain” (according to Medical Guideline for the diagnosis of interstitial cystitis). Symptoms mainly include frequent urination and nocturia, increased desire to urinate, feeling of residual urine, bladder discomfort, bladder pain, and the like. Because symptoms have various types and severity, it is not possible to specify or define the symptoms, but the symptoms are accompanied by frequent urination and bladder pain, which can greatly impair daily life. It is more common in middle-aged women but also found in men and children. It is sometimes called bladder pain syndrome.

Causes of interstitial cystitis have been considered to include dysfunction of the bladder mucosa, abnormal immunological reactions, toxic substances in urine, and hypersensitivity to pain, and the like, but the causes are still unknown. Although some documents refer to candidates of therapeutic agents (see for example, WO2011/111770), there is no internationally established treatment method, and treatment remains symptomatic. As the symptomatic treatment, explanation of the disease state and dietary guidance are used. Examples of oral drugs include analgesics, antidepressants, antiallergic drugs, immunosuppressive drugs, and the like. Repeated relapses and remissions require long-term medical management.

With regard to diagnostic criteria, the Japanese and East Asian clinical practice guidelines propose three requirements: symptoms, cystoscopic finding, and denial of other similar diseases. The cystoscopy finding in the requirements of diagnosis is Hunner lesion (characteristic reddened mucosa lacking normal capillary structures) or petechial hemorrhage after bladder hydrodistension (for details, see “Clinical Guideline for Interstitial Cystitis”). However, there are still no internationally recognized diagnostic criteria. In view of objective evaluation, diagnosis is difficult because diagnosis depends on symptoms. Furthermore, some physicians are not even able to make a diagnosis of interstitial cystitis.

### SUMMARY OF THE INVENTION

### Technical Problems

Then, an object of the present invention is to provide new means for diagnosing interstitial cystitis.

### Solution to Problem

In order to solve the above-mentioned problem, a diagnostic method of the present invention includes measuring lysophospholipid, γ-glutamyl amino acid, monoacylglycerol, free fatty acid, or lysophosphatidylethanolamine in the blood, serum, or plasma for the purpose of diagnosing interstitial cystitis. Measurement includes measuring of at least one compound, but also includes measuring of a plurality of compounds for determination.

Furthermore, the lysophospholipid is suitably lysophosphatidylcholine. Lysophospholipid is a phospholipid in which one of the two acyl groups of the phospholipids is lost. Lysophosphatidylcholine (LPC) is a derivative in which one of the fatty acids of phosphatidylcholine (lecithin) is hydrolyzed, and is also called lysolecithin. In addition, the lysophosphatidylcholine suitably includes 1-myristoyl-glycerophosphocholine, 2-myristoyl-glycerophosphocholine, 1-myristoleoyl-glycerophosphocholine, 1-oleoyl-glycerophosphocholine, 1-linoleoyl-glycerophosphocholine, 2-linoleoyl-glycerophosphocholine, 1-linolenoyl-glycerophosphocholine, 2-linolenoyl-glycerophosphocholine, and 1-eicosadienoyl-glycerophosphocholine. In particular, linoleoylglycerophosphocholine (1-linoleoylglycerophosphocholine (1-linoleoyl-GPC), and 2-linoleoylglycerophosphocholine (2-linoleoyl-GPC)). Glycerophosphocholine is one type of naturally existing choline derivative found in the brain and milk. It is a precursor of acetylcholine that acts on the parasympathetic nerves.

Furthermore, the γ-glutamyl amino acid suitably includes γ-glutamylalanine, γ-glutamylglutamic acid, γ-glutamylglutamine, γ-glutamylhistidine, γ-glutamylisoleucine, γ-glutamylleucine, γ-glutamylmethionine, γ-glutamylthreonine, γ-glutamylvaline, or γ-glutamyl-2-aminobutyric acid. Herein, γ-glutamyl amino acid indicates a γ-glutamylated amino acid. In particular, γ-glutamyl glutamic acid, γ-glutamylglutamine, γ-glutamylisoleucine, γ-glutamylvaline, and γ-glutamyl-2-aminobutyric acid are suitable.

Furthermore, the monoacylglycerol suitably includes 1-linoleoylglycerol, 1-linolenoylglycerol, and arachidonoylglycerol. In particular, 1-arachidonoylglycerol is suitable. Herein, the monoacylglycerol, also called monoglyceride, is a lipid having a structure in which one fatty acid is ester bonded to the hydroxy group of glycerol.

Furthermore, the free fatty acid suitably includes heptadecenoic acid, oleic acid, vaccenic acid, nonadecenoate, docosapentaenoic acid, docosahexaenoic acid, linoleic acid, linolenic acid, dihomolinolenic acid, arachidonic acid, docosapentaenoic acid, dihomolinoleic acid, propionylcarnitine, hydroxybutyric acid, hydroxydecanoic acid, and hydroxylaurate. In particular, propionylcarnitine is suitable.

Furthermore, the lysophosphatidylethanolamine suitably includes margaroylglycerophosphoethanolamine, 1-oleoyl-glycerophosphoethanolamine, 2-oleoyl-glycerophosphoethanolamine, 1-linoleoyl-glycerophosphoethanolamine, and 2-linoleoyl-glycerophosphoethanolamine. The lysophosphatidylethanolamine is an analogue of phosphatidylethanolamine that is a phospholipid existing in a cell membrane, and phosphatidylethanolamine is converted into lysophosphatidylethanolamine in a living body by the action of phospholipase A2 as a phospholipid hydrolase, in which one fatty acid located at sn-2 position is removed.

Furthermore, it is suitable that the measurement is performed by liquid chromatography-mass spectrometry. In addition, the present invention includes a diagnostic agent for interstitial cystitis, containing a concentration measuring reagent for lysophospholipid, γ-glutamyl amino acid, monoacylglycerol, free fatty acid, or lysophosphatidylethanolamine in the blood, serum, or plasma.

The present inventors have carried out exhaustive analysis with respect to blood of healthy subjects and patients with interstitial cystitis by liquid chromatography-mass spectrometry, resulting in finding diagnostic indicator candidates in the blood, and have completed the present invention. Conventionally, diagnostic indicators have been studied using bladder urothelium (collection is invasive) and urine, but none of them have yet been practically used. There have been no reports focusing on lipids and the like, using blood, and none of the above-described methods and substances have been mentioned as diagnostic indicators and methods for interstitial cystitis. Furthermore, the present invention enables objective diagnosis and facilitates the diagnosis itself because collection is easy and versatility is high as an examination to be performed in daily clinical practice, and the determination method and the indicator do not depend on symptoms of patients. Diagnosis from serum or plasma can be also carried out. Then, the concentrations of lysophosphatidylcholine and lysophosphatidylethanolamine in the blood is lower, and the concentrations of free fatty acid, monoacylglycerol, and γ-glutaramino acid are higher in patients with interstitial cystitis than in healthy subjects.

Phospholipid forms a bilayer to form cell membranes. The reason why lysophosphatidylcholine and lysophosphatidylethanolamine are reduced is assumed that abnormality of the urothelial epithelial maintenance and regeneration mechanism is reflected because Hunner type interstitial cystitis has characteristics that include dropout of a part of the bladder urothelium, which causes pain.

The γ-glutamyl amino acid is responsible for a variety of metabolic pathways, including synthesis of leukotriene (produced by mast cells and leukocytes and being responsible for inflammation), synthesis of glutathione (acting for protecting cells from reactive oxygen), and amino acid transport. Therefore, increase of γ-glutamyl amino acid with a decrease of glutathione metabolites in patients with interstitial cystitis is assumed to reflect enhancement of inflammation and increase in oxidative stress.

Arachidonoylglycerol (AG), as an example of a monoacylglycerol, is an endogenous ligand for the cannabinoid receptor. The affinity of 2-AG for cannabinoid receptors is 10 to 100 times that of 1-AG. On the other hand, 2-AG is unstable and rapidly isomerizes to 1-AG. The increase in AG is assumed to reflect increase in production of endocannabinoids to relieve pain due to interstitial cystitis. Furthermore, the reason of increase in free fatty acid is assumed to be because of increase in fatty acids as a decomposition product of monoacylglycerol, which is increased in interstitial cystitis.

For component analysis, chromatography-mass spectrometry is an analytical method in which gas, liquid, or supercritical fluid is used as a mobile phase, and a mixture is separated and detected by the interaction of a substance and a stationary phase held in a column. Each component in the sample is separated, and the content and a content ratio can be known. Gas chromatography-mass spectrometry using gas as a mobile phase can be applied to volatile substances, while liquid chromatography-mass spectrometry can be applied to substances from volatile substances to hardly volatile substances. The high performance liquid chromatography-mass spectrometry of the liquid chromatography-mass spectrometry is characterized in that a liquid pressurized at high pressure is used as a mobile phase. By forcing a high pressure, the mobile phase solvent is allowed to pass through the column at a high flow rate. This shortens the time for the analyte to remain on the stationary phase, and increases the resolution and detection sensitivity. Appropriate chromatographic analyses can be selected from these chromatographic analyses depending on the target substance. Furthermore, not only one compound but also a plurality of compounds can be measured and the measured compounds can be combined for determination.

Furthermore, when a diagnostic agent containing a concentration measurement reagent using a specific receptor of the above-mentioned substance is provided, it can be provided as an effective kit for diagnosing interstitial cystitis.

### BRIEF DESCRIPTION OF THE DRAWINGS

In the drawings:

FIG. 1 is a plot of the content of 1-linoleoylglycerophosphocholine in the blood (μg/mL) in 25 healthy subjects and 25 patients with interstitial cystitis with Hunner lesion;

FIG. 2 is a plot of an ROC curve for the data of FIG. 1;

FIG. 3 is a plot of a comparison of the healthy subjects and patients with interstitial cystitis with Hunner lesion of FIG. 1 compared by age and shows an example of the cutoff value in which the sensitivity and the specificity are balanced;

FIG. 4 is a plot of the ratio (weight ratio) of 1-linoleoylglycerophosphocholine to phospholipid for 25 healthy subjects and 25 patients with interstitial cystitis with Hunner lesion;

FIG. 5 is a plot of an ROC curve for the data of FIG. 4; and

FIG. 6 is a plot of a comparison of the healthy subjects and patients with interstitial cystitis with Hunner lesion of FIG. 4 compared by age and shows an example of the cutoff value in which both the sensitivity and the specificity are balanced.

### ADVANTAGEOUS EFFECTS OF INVENTION

The present invention enables an objective, simple and clear diagnosis or determination of interstitial cystitis to be made. Therefore, the present invention is useful not only for initial screening, early diagnosis, and early start of treatment, but also for development of therapeutic agents for interstitial cystitis. In diagnosis, the present invention is also easily combined with symptoms, cystoscopic findings, denial of other similar diseases, and the like. Thus, the present invention is useful in helping patients with interstitial cystitis who are overlooked.

### DESCRIPTION OF EMBODIMENTS

The sensitivity and specificity, likelihood ratio, P-value, and content (increase/decrease relative to that of healthy subjects) of each substance for interstitial cystitis, as determined by liquid chromatography-mass spectrometry, are shown in the following Tables, and all of them have been demonstrated to be useful for diagnosis (population (blood): 10 healthy subjects and 20 patients with interstitial cystitis).

TABLE 1

| LPC: | | | | | | Relative to |
|---|---|---|---|---|---|---|
| lysophosphatidylcholine | | SE | SP | LI | P-value | healthy subject |
| 1-myristoyl-GPC (14:0) | 1-myristoyl- | 55 | 90 | 5.5 | 0.0073 | Reduce |
| | glycerophosphocholine | | | | | |
| 2-myristoyl-GPC (14:0) | 2-myristoyl- | 60 | 90 | 5.5 | 0.0122 | Reduce |
| | glycerophosphocholine | | | | | |
| 1-myristoleoyl | 1-myristoleoyl | 60 | 90 | 6 | 0.0197 | Reduce |
| glycerophosphocholine | glycerophosphocholine | | | | | |
| (14:1) | | | | | | |
| 1-oleoyl-GPC (18:1) | 1-oleoyl- | 70 | 90 | 7 | 0.0024 | Reduce |
| | glycerophosphocholine | | | | | |
| 1-linoleoyl-GPC (18:2) | 1-linoleoyl- | 75 | 90 | 7.5 | 0.0024 | Reduce |
| | glycerophosphocholine | | | | | |
| 2-linoleoyl-GPC (18:2) | 2-linoleoyl- | 75 | 90 | 7.5 | 0.0013 | Reduce |
| | glycerophosphocholine | | | | | |
| 1-linolenoyl- | 1-linolenoyl- | 80 | 90 | 8 | 0.0007 | Reduce |
| glycerophosphocholine | glycerophosphocholine | | | | | |
| (18:3n3) | | | | | | |
| 2-linolenoyl- | 2-linolenoyl- | 70 | 90 | 7 | 0.0083 | Reduce |
| glycerophosphocholine | glycerophosphocholine | | | | | |
| (18:3n3) | | | | | | |
| 1-eicosadienoyl-GPC (20:2) | 1-eicosadienoyl- | 55 | 90 | 5.5 | 0.0028 | Reduce |
| | glycerophosphocholine | | | | | |

SE = Sensitivity;
SP = Specificity;
LI = Likelihood
(The same is true in the following Tables)

TABLE 2

| | | | | | P- | Relative to |
|---|---|---|---|---|---|---|
| γ-glutamyl amino acid | | SE | SP | LI | value | healthy subject |
| gamma- | γ-glutamylalanine | 60 | 90 | 6 | 0.0073 | Increase |
| glutamylalanine | | | | | | |
| gamma- | γ-glutamyl glutamic | 70 | 90 | 7 | 0.0021 | Increase |
| glutamylglutamate | acid | | | | | |
| gamma- | γ-glutamylglutamine | 80 | 90 | 8 | 0.0007 | Increase |
| glutamylglutamine | | | | | | |
| gamma- | γ-glutamylhistidine | 45 | 90 | 4.5 | 0.0823 | Increase |
| glutamylhistidine | | | | | | |
| gamma- | γ-glutamylisoleucine | 70 | 90 | 7 | 0.0056 | Increase |
| glutamylisoleucine | | | | | | |
| gamma- | γ-glutamylleucine | 60 | 90 | 6 | 0.0529 | Increase |
| glutamylleucine | | | | | | |
| gamma- | γ-glutamylmethionine | 60 | 90 | 6 | 0.0042 | Increase |
| glutamylmethionine | | | | | | |
| gamma- | γ-glutamylthreonine | 55 | 90 | 5.5 | 0.1405 | Increase |
| glutamylthreonine | | | | | | |
| gamma- | γ-glutamylvaline | 80 | 90 | 8 | 0.0008 | Increase |
| glutamylvaline | | | | | | |
| gamma-glutamyl-2- | γ-glutamyl-2- | 85 | 90 | 8.5 | 0.0006 | Increase |
| aminobutyrate | aminobutyric acid | | | | | |

TABLE 3

| MAG: | | | | | | Relative to |
|---|---|---|---|---|---|---|
| Monoacylglycerol | | SE | SP | LI | P-value | healthy subject |
| 1-linoleoylglycerol | 1-linoleoylglycerol | 60 | 90 | 6 | 0.0249 | Increase |
| (18:2) | | | | | | |
| 1-linolenoylglycerol | 1-linolenoylglycerol | 45 | 90 | 4.5 | 0.1183 | Increase |
| 1-arachidonoylglycerol | 1-arachidonoylglycerol | 75 | 90 | 7.5 | 0.0028 | Increase |

TABLE 4

| | | | | | | Relative to |
|---|---|---|---|---|---|---|
| FFA: free fatty acid | | SE | SP | LI | P-value | healthy subject |
| 10-heptadecenoate | heptadecenoic acid | 55 | 90 | 5.5 | 0.1036 | Increase |
| (17:1n7) | | | | | | |
| oleate (18:1n9) | oleic acid | 40 | 90 | 4 | 0.1236 | Increase |
| vaccinate (18:1n7) | vaccenic acid | 40 | 90 | 4 | 0.1726 | Increase |
| 10-nonadecenoate | nonadecenoate | 60 | 90 | 6 | 0.1236 | Increase |
| (19:1n9) | | | | | | |
| docosapentaenoate | docosapentaenoic | 45 | 90 | 4.5 | 0.1132 | Increase |
| (DPA: 22:5n3) | acid | | | | | |
| docosahexaenoate | docosahexaenoic | 45 | 90 | 4.5 | 0.1036 | Increase |
| (DHA: 22:6n3) | acid | | | | | |
| linoleate (18:2n6) | linoleic acid | 45 | 90 | 4.5 | 0.1466 | Increase |
| linolenate (18:3n3 or | linolenic acid | 45 | 90 | 4.5 | 0.1869 | Increase |
| 3n6) | | | | | | |
| dihomolinolenate | dihomolinolenic | 50 | 90 | 5 | 0.1869 | Increase |
| (20:3n3 or 3n6) | acid | | | | | |
| arachidonoate (20:4n6) | arachidonic acid | 60 | 90 | 6 | 0.0646 | Increase |
| docosapentaenoate | docosapentaenoic | 45 | 90 | 4.5 | 0.0784 | Increase |
| (n6 DPA: 22:5n6) | acid | | | | | |
| dihomolinoleate | dihomolinoleic acid | 50 | 90 | 5 | 0.1466 | Increase |
| (20:2n6) | | | | | | |
| propionylcarnitine (C3) | propionylcarnitine | 75 | 90 | 7.5 | 0.0073 | Increase |
| 3-hydroxybutyrate | hydroxybutyric acid | 50 | 90 | 5 | 0.0946 | Increase |
| (BHBA) | | | | | | |
| 3-hydroxydecanoate | hydroxydecanoic | 70 | 90 | 7 | 0.0197 | Increase |
| | acid | | | | | |
| 3-hydroxylaurate | hydroxylaurate | 65 | 90 | 6.5 | 0.0107 | Increase |

TABLE 5

| LPE: | | | | | | |
|---|---|---|---|---|---|---|
| Lysophosphatidyl- | | | | | P- | Relative to |
| ethanolamine | | SE | SP | LI | value | healthy subject |
| 1-margaroyl | Margaroylglycero- | 65 | 90 | 6.5 | 0.0008 | Reduce |
| Glycerophospho- | phosphoethanolamine | | | | | |
| ethanolamine | | | | | | |
| 1-oleoyl-GPE (18:1) | 1-oleoyl-glycerophospho- | 60 | 90 | 6 | 0.0122 | Reduce |
| | ethanolamine | | | | | |
| 2-oleoyl-GPE (18:1) | 2-oleoyl-glycerophospho- | 65 | 90 | 6.5 | 0.0122 | Reduce |
| | ethanolamine | | | | | |
| 1-linoleoyl-GPE (18:2) | 1-linoleoyl-glycerophospho- | 40 | 90 | 4 | 0.0138 | Reduce |
| | ethanolamine | | | | | |
| 2-linoleoyl-GPE (18:2) | 2-linoleoyl-glycerophospho- | 50 | 90 | 5 | 0.0064 | Reduce |
| | ethanolamine | | | | | |

In the above, 1-linoleoyl-glycerophosphocholine, 2-linoleoyl-glycerophosphocholine, 1-linolenoyl-glycerophosphocholine, 2-linolenoyl-glycerophosphocholine, 1-linoleoylglycerophosphocholine, 2-linoleoylglycerophosphocholine, γ-glutamyl glutamic acid, γ-glutamylglutamine, γ-glutamylisoleucine, γ-glutamylvaline, γ-glutamyl-2-am inobutyric acid, 1-arachidonoylglycerol, and propionylcarnitine have sensitivity of 70% or more, specificity of 90% or more, likelihood ratio of 7 or more, and P value in the level of 1%, and are effective and further suitable for diagnosis for interstitial cystitis.

Furthermore, the contents of γ-glutamylisoleucine, 1-linoleoyl-glycerophosphocholine, and 1-arachidonoylglycerol in the blood in 5 patients with interstitial cystitis and 5 healthy subjects (see Table 6 below) were determined by high-performance chromatography-mass spectrometry.

TABLE 6

| Interstitial cystitis | Healthy subjects |
|---|---|
| patient group | group |
| Hunner type: age 80 | age: 22 |
| Hunner type: age 42 | age: 25 |
| Hunner type: age 68 | age: 22 |
| Hunner type: age 67 | age: 23 |
| non-Hunner type: age 34 | age: 25 |

The results are shown in the following Tables 7-9.

TABLE 7
γ-glutamylisoleucine [average ± standard deviation]

| Patient with | Healthy | 5 subjects |
|---|---|---|
| interstitialcystitis | subjects | each |
| 13.50 ± 4.50 | 9.974 ± 2.962 | Unit: ng/mL |

TABLE 8
1-linoleoylglycerophosphocholine [average ± standard deviation]

| Patient with | Healthy | 5 subjects |
|---|---|---|
| interstitialcystitis | subjects | each |
| 20020 ± 7008 | 30980 ± 9800 | Unit: ng/mL |

TABLE 9
1-arachidonoylglycerol [average ± standard deviation]

| Patient with | Healthy | 5 subjects |
|---|---|---|
| interstitialcystitis | subjects | each |
| 0.5456 ± 0.3383 | 0.2840 ± 0.1242 | Unit: ng/mL |

As mentioned above, the patients with interstitial cystitis have higher concentrations of γ-glutamylisoleucine and 1-arachidonoylglycerol in the blood and lower concentrations of 1-linoleoylglycerophosphocholine in the blood as compared with the healthy subjects. In addition, since 1-linoleoylglycerophosphocholine is overwhelmingly large in the detected amount per unit, it is assumed that linoleoylglycerophosphocholine is advantageous in cost and efficiency in the detection.

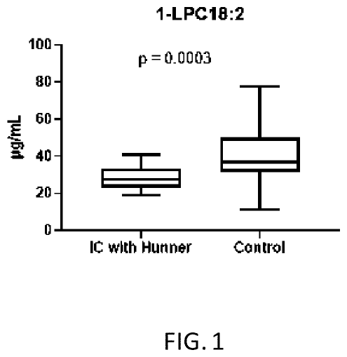

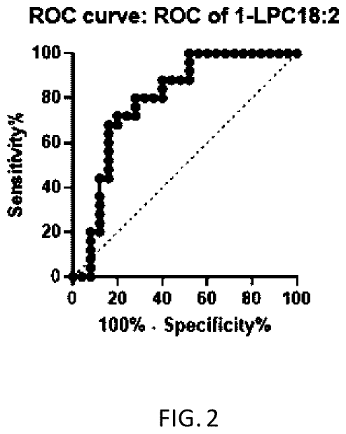

From the above results, the content of 1-linoleoylglycerophosphocholine in the blood (μg/mL) in 25 healthy subjects and 25 patients with interstitial cystitis with Hunner lesion were obtained and compared in order to seek the criteria for facilitating determination of interstitial cystitis (see FIG. 1). An ROC curve was also calculated (see FIG. 2).

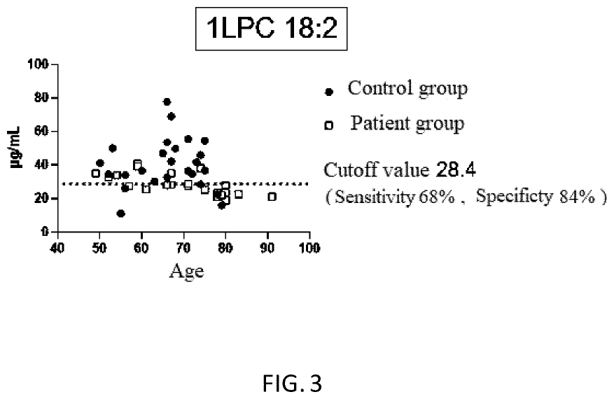

Furthermore, both were compared by age. FIG. 3 shows an example of the cutoff value in which the sensitivity and the specificity are balanced.

As shown in FIGS. 1-3, the content of 1-linoleoylglycerophosphocholine can be one of the indicators for diagnosis. In other words, if the content of a predetermined cutoff value or less, it can be understood that interstitial cystitis is suspected. Therefore, when the content is used as an indicator at the initial stage determining the necessity of cystoscopy. From the above results, the cutoff value can be 20 or more and less than 40, but more preferably 25 to 35 from the viewpoint that the balance between the sensitivity and the specificity is desirable. For example, when the cutoff value is 40, the sensitivity is increased but the specificity is largely reduced.

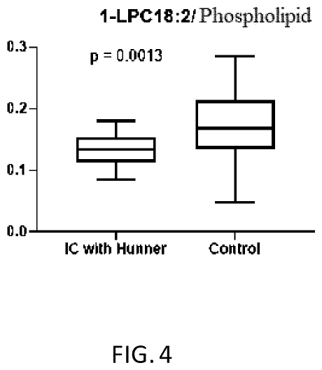

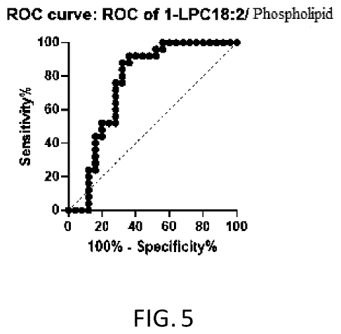

In addition, based on the above findings, a possibility that examining the ratio with respect to phospholipid in subjects is useful was found, and experiments were carried out. Specifically, the ratio (weight ratio) of 1-linoleoylglycerophosphocholine to phospholipid was obtained for 25 healthy subjects and 25 patients with interstitial cystitis with Hunner lesion and compared (see FIG. 4). An ROC curve was also calculated (see FIG. 5).

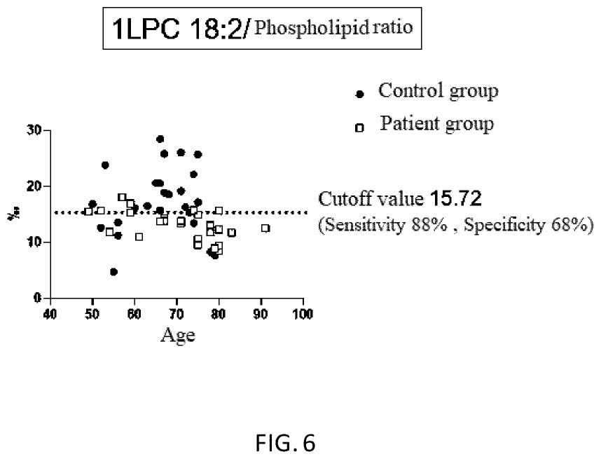

In addition, both were compared by age. FIG. 6 shows an example of the cutoff value in which both the sensitivity and the specificity are balanced.

As shown above, when determination is carried out based on the ratio of 1-linoleoylglycerophosphocholine to phospholipid, it is found that that the indicator has higher sensitivity and specificity as compared with the case where determination is carried out using a simple substance of 1-linoleoylglycerophosphocholine as the indicator. The cutoff value seems to be desirably 10 or more and less than 20, and particularly desirably 15 or more and less than 16 in view of the balance between the sensitivity and specificity. When the cutoff value is, for example, 20, the sensitivity is increased, but the specificity is reduced.

Treatment strategies for interstitial cystitis vary widely depending on the presence or absence of Hunner lesion. Cystoscopy is necessary for the diagnosis of Hunner lesion, but it is important to accurately determine the need for it because of the psychological and physical burden on patients.

As mentioned above, the ratio of 1-linoleoylglycerophosphocholine to phospholipid can be an indicator (in particular, suspicion indicator) of “interstitial cystitis”. In addition, high sensitivity and specificity are achieved. In the current situation where the diagnosis of interstitial cystitis itself is not reached so often, the ratio is particularly useful as an early diagnostic indicator.

The present invention can be made as a system or a program using the above-mentioned compound or the indicator as a criterion. In other words, the present invention can be made as a system or a program having means for receiving an input of a value (one type or more values) obtained from a content of lysophospholipid, γ-glutamyl amino acid, monoacylglycerol, free fatty acid, or lysophosphatidylethanolamine included in the blood, serum, or plasma; comparing a predetermined threshold with the input value; and determining whether the input value is higher or lower than the predetermined threshold, wherein the predetermined value is a value applicable to diagnosis of interstitial cystitis. The threshold is not limited to a particular numerical value as long as it is a value useful as an indicator for diagnosis for interstitial cystitis, in view of interstitial cystitis and feature of the present invention, particularly, it is preferable to select a value useful as an indicator for initial diagnosis.

Specifically, when the content, for example (μg/mL), of the above-mentioned compounds, preferably 1-linoleoylglycerophosphocholine, and more preferably a ratio (weight ratio) of 1-linoleoylglycerophosphocholine to phospholipid in the blood, serum, or plasma of subjects, are measured, and the measured value is input, if the value is the predetermined threshold or less (threshold or more depending on compound), suspicion of interstitial cystitis is determined and output. In addition, it is also possible to configure a system or a program in which a plurality of values is provided and the values are in a predetermined range, the values are categorized in stages to output a possibility. Specifically, it is possible to employ a configuration in which a plurality of threshold values is provided, and the possibility is determined in stages. For example, in the case of 5 stages, the determination may be categorized from A (high) to E (low).

1-23. (canceled)

24. A method for diagnosing interstitial cystitis, the method comprising measuring at least any one kind of lysophosphatidylcholine.

25. The method according to claim 24, wherein the method comprises measuring any one or more of 1-myristoyl-glycerophosphocholine, 2-myristoyl-glycerophosphocholine, 1-myristoleoylglycerophosphocholine, 1-oleoyl-glycerophosphocholine, 1-linoleoyl-glycerophosphocholine, 2-linoleoyl-glycerophosphocholine, 1-linolenoyl-glycerophosphocholine, 2-linolenoyl-glycerophosphocholine, and 1-eicosadienoyl-glycerophosphocholine.

26. The method according to claim 24, wherein the method comprises measuring any one or more of 1-linoleoyl-glycerophosphocholine, 2-linoleoyl-glycerophosphocholine, 1-linolenoyl-glycerophosphocholine, and 2-linolenoyl-glycerophosphocholine.

27. The method according to claim 24, wherein the measuring is a measurement by chromatography-mass spectrometry.

28. The method according to claim 24, comprising measuring a ratio of 1-linoleoylglycerophosphocholine to phospholipid in blood, serum, or plasma.

29. A system, the system configured to:
receive an input of a value obtained from a content of at least any one kind of lysophosphatidylcholine included in the blood, serum, or plasma;
compare a predetermined threshold with the input value; and
determine whether the input value is higher or lower than the predetermined threshold, wherein the predetermined threshold is a value for diagnosing a possibility of interstitial cystitis or bladder pain syndrome.

30. The system according to claim 29, wherein the system is configured to:
receive an input of a value obtained from a content of at least any one of 1-myristoyl-glycerophosphocholine, 2-myristoyl-glycerophosphocholine, 1-myristoleoylglycerophosphocholine, 1-oleoyl-glycerophosphocholine, 1-linoleoyl-glycerophosphocholine, 2-linoleoyl-glycerophosphocholine, 1-linolenoyl-glycerophosphocholine, 2-linolenoyl-glycerophosphocholine, and 1-eicosadienoyl-glycerophosphocholine included in the blood, serum, or plasma;
compare a predetermined threshold with the input value; and
determine whether the input value is higher or lower than the predetermined threshold, wherein the predetermined threshold is a value for diagnosing a possibility of interstitial cystitis or bladder pain syndrome.

31. The system according to claim 30, wherein an indicator of a possibility of interstitial cystitis or bladder pain syndrome is a value obtained from a content of 1-linoleoylglycerophosphocholine.

32. The system according to claim 29, wherein an indicator of a possibility of interstitial cystitis or bladder pain syndrome is a ratio of 1-linoleoylglycerophosphocholine to phospho lipid.

33. The system according to claim 29, wherein the system is configured to determine a possibility of interstitial cystitis or bladder pain syndrome in stages with a plurality of thresholds.

34. A program for a computer, wherein the program is configured to control the computer to:
receive a value obtained from a content of at least any one kind of lysophosphatidylcholine included in the blood, serum, or plasma, and a predetermined threshold;
compare the predetermined threshold with the value; and
determine whether the value is higher or lower than the predetermined threshold, wherein the predetermined threshold is a value for diagnosing a possibility of interstitial cystitis or bladder pain syndrome.

35. The program according to claim 34, wherein the program is configured to control the computer to:
receive a value obtained from a content of at least any one kind of 1-myristoyl-glycerophosphocholine, 2-myristoyl-glycerophosphocholine, 1-myristoleoylglycerophosphocholine, 1-oleoyl-glycerophosphocholine, 1-linoleoyl-glycerophosphocholine, 2-linoleoyl-glycerophosphocholine, 1-linolenoyl-glycerophosphocholine, 2-linolenoyl-glycerophosphocholine, and 1-eicosadienoyl-glycerophosphocholine included in the blood, serum, or plasma, and a predetermined threshold;
compare the predetermined threshold with the value; and
determine whether the value is higher or lower than the predetermined threshold, wherein the predetermined threshold is a value for diagnosing a possibility of interstitial cystitis or bladder pain syndrome.

36. The program according to claim 34, wherein an indicator of a possibility of interstitial cystitis or bladder pain syndrome is a value obtained from the content of 1-linoleoylglycerophosphocholine.

37. The program according to claim 34, wherein an indicator of possibility of interstitial cystitis or bladder pain syndrome is a value of a ratio of 1-linoleoylglycerophosphocholine to phospholipid.

38. The program according to claim 34, wherein the program is configured to determine a possibility of interstitial cystitis or bladder pain syndrome in stages with a plurality of thresholds.
